(19) United States
(12) Patent Application Publication
(10) Pub. No.: US 2024/0108252 A1
(43) Pub. Date: Apr. 4, 2024

(54) DETECTION MECHANISM FOR A MEDICAL SENSING TOOL, MEDICAL SENSING TOOL

(71) Applicant: ECOLE POLYTECHNIQUE FEDERALE DE LAUSANNE, Lausanne (CH)

(72) Inventors: Loic Tissot-Daguette, Neuchatel (CH); Charles Baur, Neuchatel (CH); Hubert Schneegans, Neuchatel (CH); Axel Bertholds, Losone (CH); Pere Llosas, Losone (CH)

(73) Assignee: ECOLE POLYTECHNIQUE FEDERALE DE LAUSANNE, Lausanne (CH)

(21) Appl. No.: 18/527,752

(22) Filed: Dec. 4, 2023

Related U.S. Application Data
(63) Continuation of application No. PCT/EP2022/064711, filed on May 31, 2022.

(30) Foreign Application Priority Data
Jun. 4, 2021 (EP) 211778402

(51) Int. Cl. A61B 5/12; A61B 5/00

(52) U.S. Cl. CPC A61B 5/125; A61B 5/6817; A61B 5/6847

(57) ABSTRACT

A micromachined mechanism for a sensing tool includes a support, a probe pivotally mounted with respect to the support, and a beam connected to the support and the probe. The probe is configured to apply a load (Fₜₒₒₗ) on a body and to be subjected to a counter-reaction contact force exerted by the body in reaction to the load. The beam is configured to shift from an undeformed position to a deformed position when the contact force of the body is greater than a predetermined threshold force (F_{crit}) for which the beam buckles.

### CROSS-REFERENCE TO RELATED APPLICATIONS

This application is a continuation of International Application No. PCT/EP2022/064711, filed on May 31, 2022, which claims priority to and the benefit of EP 21177840.2 filed on June 4, 2021. The disclosures of the above applications are incorporated herein by reference.

### FIELD

The present disclosure relates to the medical field and a sensor device for sensing a body, but not exclusively, and a micromachined mechanism for such sensor device.

### BACKGROUND

The statements in this section merely provide background information related to the present disclosure and may not constitute prior art.

A micromachined mechanism refers to a mechanism that has at least one dimension less than one-hundred micrometers.

During the performance of medical or surgical procedures, the application and transmission of an uncontrolled load by the surgeon may ultimately result in undesirable effects to the tissues being handled. Therefore, the control of the applied force during medical or surgical acts is key for a successful and improved surgery.

The middle ear plays a crucial role in hearing, which functions to transmits the sounds from the outer ear to the inner ear, where sound can be communicated to the brain. Sound waves are ducted into the outer ear, and then strike the tympanic membrane causing the tympanic membrane to vibrate. These vibrations are transmitted via three bones called ossicles or ossicle chain: the malleus, the incus and the stapes. The sound strikes an oval window, which separates the middle ear from the inner ear. When the oval window is hit, the impact causes waves in the fluid inside the inner ear and sets into motion a chain of events leading to the interpretation of sound by the brain.

Pathologies such as otosclerosis and sequels of chronic otitis media may cause fixation of the middle ear ossicles, leading to hearing impairment. Knowledge of the degree of ossicular mobility is useful to determine the best course of surgical treatment.

Sound transmission can be estimated by evaluating the stiffness, also named mobility, of each ossicle.

Otologists usually carry out such assessment by manual palpation with a stiff tool. However, the palpation forces are in the order of 3-15 gf (1 gf corresponding to 9.81 mN).

Thereby, the judgment of normal or impaired mobility of the middle ear ossicles is highly dependent on the subjective experience of a surgeon. Moreover, such assessment of mobility is imprecise.

Sensors have been proposed to enable a quantitative measure of the ossicular mobility.

U.S. Patent Publication No. 20130204142 A1 (Sensoptic) discloses an optical force-sensing instrument to measure the force applied to the ossicles. Such force measurement is based on the ossicle displacement, visualized under an operating microscope.

International Patent Publication No. WO 2015/168698 A1 (University of Colorado) discloses a measuring tool that uses a linear actuator coupled to a highly sensitive load cell to measure ossicle compliance.

Another reference proposes a vibrating probe that administers an oscillating displacement on the ossicles and measures the contact force with a load cell.

Although the above presented devices are considered to be effective to carry out a quantitative measure, they have nevertheless some drawbacks. The existing devices are all active, i.e., they comprise sensors and/or actuators. Therefore, such components use electrical energy, which increases the complexity of the use of the devices, and the space provided. Hence, due to the small size, the manufacture of such active devices is complex and relatively expensive compared to common stiff hooks, which are not intended for single use.

Epiretinal membrane (ERM) is a disorder that concerns the macula. The macula, disposed in the center of the retina, contains cells that receive and analyze light signals. The information is then transmitted by the optic nerve to the brain which reconstitutes the image. The ERM consists of the proliferation of fibrocellular membranes on the inner retinal surface in the macular area. Such disorder may occur in healthy eyes, but can be secondary to retinal breaks, or rhegmatogenous retinal detachment, retinal vascular diseases, intraocular inflammation, blunt or penetrating trauma, and other ocular disorders. A presence of ERM progressively affects the central vision and can cause metamorphopsia, that lessens the visual acuity.

ERM is common and is present in approximately 10% of people over the age of 70 years.

The ERM can be treated by surgery, with the surgery technique of vitrectomy combined with the removal of the ERM by peeling.

The peeling of the ERM is a delicate procedure since the retina must not be damaged, the principal difficulty being the limitation of human performance at a required millinewton force range.

To overcome these challenges, a mecano-optical transducer has been developed. After removal of the vitreous gel of the eye, the instrument is inserted into the eye, and peels away the ERM. The instrument comprises a force sensor integrating a flexure mechanism and performing an interferometric measurement, notably via an optical fiber of the load applied by the surgeon during peeling. Force sensing is indicated by increasing frequency sounds as the force approaches the maximal limit, and a warning sound when above. The applied force is also recorded in real time and displayed on a screen, so an assistant can inform the surgeon of the measured force.

However, such instruments use an external source of electric energy. Moreover, such instrument is not intended to single-use, and therefore must undergo a sterilization protocol after use.

### SUMMARY

This section provides a general summary of the disclosure and is not a comprehensive disclosure of its full scope or all of its features.

The present disclosure relates to a micromachined mechanism for a sensing tool intended for a medical or surgical use. The mechanism comprises a support, a probe pivotally mounted with respect to the support, and a beam connected to the support and the probe. The probe is intended to apply a load on a body, and to be subjected to a counter-reaction contact force exerted by the body in reaction to the load. The beam is configured to shift from an undeformed position to a deformed position when the contact force of the body is greater than a predetermined threshold force for which the beam buckles.

The mechanism facilitates the application of a constant load on a body, without an external source of electric energy. The compact and simple structure facilitates economic manufacturing, which facilitates a single use of a sensing tool equipped with the mechanism.

In some forms, the probe is pivotally mounted with respect to the support via a flexure pivot.

In some variations, the probe is pivotally mounted with respect to the support via a pivot connection comprising a pin, and a torsion spring.

In some forms, the mechanism is monolithic.

Further, the mechanism is made of one of the materials selected from the group consisting of: metal, silicon, glass, thermoset or thermoplastic.

In an undeformed position the beam extends along a slightly curved trajectory, the curved trajectory having a curvature radius convex in view of the probe.

In some variations, the probe comprises the extreme portion comprising a tip intended to be in contact with the body.

In some variations, the probe comprises a connection part and an extreme portion, the extreme portion extending substantially in a direction that is secant with the direction of extension of the connection part.

In some forms, an end portion encompasses a graduation element. The graduation element protrudes, which can help to visually measure the displacement of the body.

Further, the graduation element comprises a three-dimensional solid such as a cylinder arranged along a surface of the end portion.

In one aspect, the mechanism is adapted for a sensing tool intended to palpate a body, the body being an ossicle.

In another aspect, the mechanism is adapted for a sensing tool intended to remove a membrane from a retina, the body being the membrane.

In an aspect, the present disclosure relates to a sensing tool for middle ear ossicles mobility assessment comprising a handle element, a holding element placed at one end of the handle element, and a force-constant mechanism. The tool is characterized in that the mechanism is rigidly connected with the holding part.

In another aspect, the present disclosure relates to a method of manufacturing of the mechanism. The method includes a step of formation carried out by femto-laser printing, or three-dimensional (3D) printing.

Further areas of applicability will become apparent from the description provided herein. It should be understood that the description and specific examples are intended for purposes of illustration only and are not intended to limit the scope of the present disclosure.

### DRAWINGS

In order that the disclosure may be well understood, there will now be described various forms thereof, given by way of example, reference being made to the accompanying drawings, in which:

FIG. 1 illustrates a side view of a sensing tool according to the present disclosure;

FIG. 2 illustrates a perspective view of a part of the sensing tool according to the present disclosure;

FIG. 3 illustrates a side view of a mechanism, the mechanism being undeformed according to the present disclosure;

FIG. 4 illustrates a perspective view of a mechanism, the mechanism being undeformed according to the present disclosure;

FIG. 5 illustrates a schematic side view of a mechanism, the mechanism being undeformed according to the present disclosure;

FIG. 6 illustrates a schematic side view of a mechanism, the mechanism being deformed according to the present disclosure;

FIG. 7 illustrates a schematic side view of an analytical model mechanism, the mechanism being undeformed according to the present disclosure;

FIG. 8 represents a plot of the force exerted by the sensing tool on a body as a function of the displacement of the sensing tool tip with respect to the sensing tool support, from undeformed to deformed position according to the present disclosure;

FIG. 9 illustrates a schematic side view of the sensing tool in use during operations for middle ear ossicles mobility assessment according to the present disclosure;

FIG. 10 illustrates a schematic side view of the sensing tool in use during operations for middle ear ossicles mobility assessment as shown in FIG. 9 according to the present disclosure;

FIG. 11 illustrates a schematic side view of the sensing tool in use during operations for middle ear ossicles mobility assessment as shown in FIG. 9 according to the present disclosure;

FIG. 12 illustrates a schematic side view of the sensing tool in use during operations for middle ear ossicles mobility assessment as shown in FIG. 9 according to the present disclosure;

FIG. 13 illustrates a schematic side view of the sensing tool in use during operations for middle ear ossicles mobility assessment as shown in FIG. 9 according to the present disclosure;

FIG. 14 illustrates a schematic side view of the sensing tool in use during ERM peeling operations according to the present disclosure; and

FIG. 15 illustrates a schematic side view of the sensing tool in use during ERM peeling operations as shown in FIG. 14 according to the present disclosure.

The drawings described herein are for illustration purposes only and are not intended to limit the scope of the present disclosure in any way.

### DETAILED DESCRIPTION

The following description is merely exemplary in nature and is not intended to limit the present disclosure, application, or uses. It should be understood that throughout the drawings, corresponding reference numerals indicate like or corresponding parts and features.

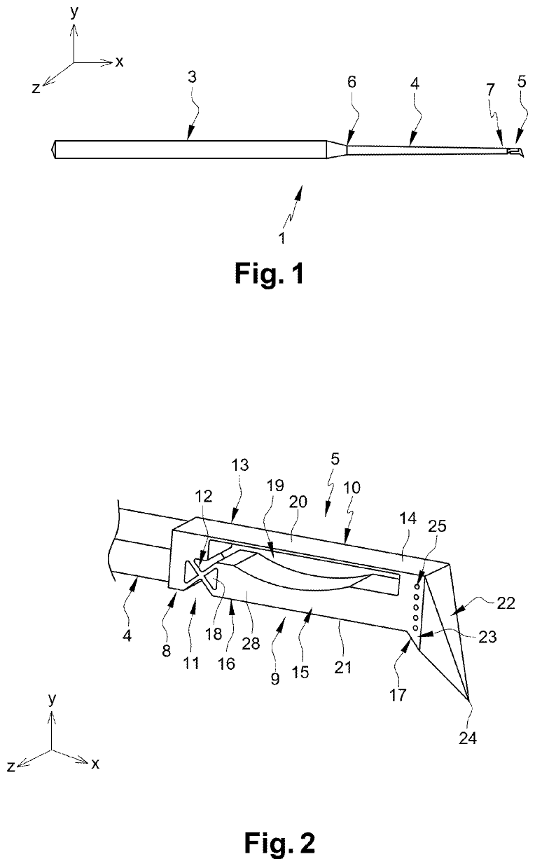

Referring to FIGS. 1 to 4, a sensing tool 1 is illustrated. The sensing tool 1 is intended to apply a force on a body and is intended for a medical or surgical use.

The sensing tool 1 comprises a handle element 3, a holding element 4 and a mechanism 5, that are connected to each other.

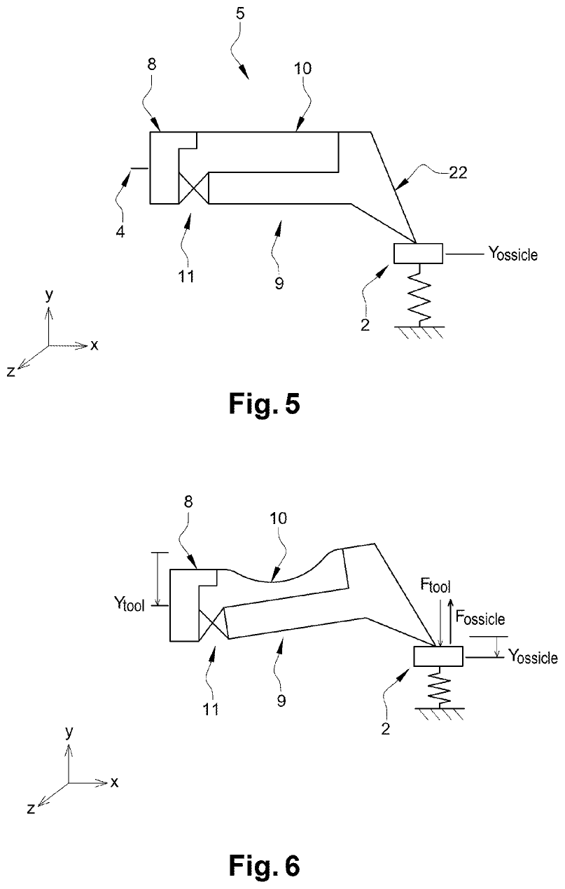

In a first use, the sensing tool 1 assists a surgeon on the assessment of the mobility of the ossicle chain on patients by palpation of one or several of the three ossicles 2, as shown in FIG. 5.

The mechanism 5 and the sensing tool 1 are described in detail for the use in ossicle chain measurement. However, the principle and the structure of the mechanism 5 and the sensing tool 1 remains the same. One skilled in the art is able to transpose the technique from a tool for palpation of the ossicles 2 to a tool for pulling an ERM. Such second use is described further in the present text.

The sensing tool 1 is considered passive, as the sensing tool 1 does not use electrical energy. The sensing tool 1 does not comprise any sensors or actuators that are electrically driven. The sensing tool 1 is thus simple to use for any surgeon, and is lighter and more compact than those already known. Hence, the sensing tool 1 does not use an energy supply.

Referring to FIGS. 1 and 9-11, the sensing tool 1 extends substantially on an elongated shape, like a rod, to occupy a reduced transversal space. As an example, the sensing tool 1 can be a dozen centimeters long, and the mechanism 5 is a few millimeters long.

As described later in the present text, and as can be seen in FIGS. 5-6 and 9 to 13, the sensing tool 1, more specifically the mechanism 5, displaces one of the ossicles 2 under a predefined threshold force F_{crit}.

The mechanism 5 applies a progressive force Fₜₒₒₗ to one of the ossicles 2 until deformed. After deformation of the mechanism 5, the force Fₜₒₒₗ exerted by the sensing tool 1 on the ossicle 2 remains nearly constant and equal to the threshold force Fcrit. The user can consequently measure the amplitude of the displacement y_{ossicle} of the ossicles 2 subjected to the constant force Fₜₒₒₗ and assess the mobility of the ossicles 2.

In the illustrated form, the sensing tool 1 is monolithic, or one-piece. Such feature renders the sterilization easy. Moreover, the sensing tool 1 can be a single-use device. Thus, the sensing tool 1 can be used and then disposed.

In other forms not shown, the mechanism 5 is assembled on the holding element 4. In such form, the mechanism 5 can be removable from the holding element 4, which facilitates the change of mechanism 5 after use. The mechanism 5 can then be disposed.

In the following description, an orthogonal reference frame XYZ is defined with three axes perpendicular to each other, namely: an X axis, defining a longitudinal, horizontal direction, coincident with a general direction of extension of the sensing tool 1; a Y axis, defining a transverse, vertical direction, which with the X axis defines a vertical XY plane; and a Z axis, defining a transverse, horizontal direction, perpendicular to the vertical XY plane.

The handle element 3 is intended to receive the hand of a user, and can be provided with a grip (not shown) that facilitates an easy handling of the sensing tool 1.

The holding element 4 is connected on a first end 6 at the handle element 3, and on a second end 7 at the holding element 4. The first end 6 defines, for example, a frustoconical shape, devoid of any protruding sharp part, or corners. The absence of such protrusions facilitates a reduced chance for harm while using sensing tool 1, and eases cleaning of the handle element 3 and the holding element 4.

With reference to FIGS. 2 and 4, the mechanism 5 comprises a support 8, a probe 9 and a beam 10. The probe 9 is pivotable with respect to the support 8. The mechanism 5 can be a micromachined structure, i.e., has one of the dimensions below a hundred micrometers.

In the illustrated forms, the probe 9 is pivotally mounted with respect to the support 8 due to a flexure pivot 11 that gives a degree of flexibility to the mechanism 5. The flexibility facilitates the probe 9 remaining in contact with the ossicles 2. The flexibility of the flexure pivot 11 is, for example, designed to adapt the general stiffness of the pivot. The flexibility facilitates the adaption of the constancy of the applied force Fₜₒₒₗ so that the force Fₜₒₒₗ is nearly constant and equal to the threshold force F_{crit} when the mechanism 5 is deformed.

In the illustrated forms, the mechanism 5 integrates a cross-spring pivot, that contains several blades 12. Such pivot linkage can be manufactured monolithically within the probe 9. The cost of production can be limited.

In other alternative forms not shown, the mechanism 5 can comprise a shaft that links the support 8 and the probe 9, and a balance spring aligned with the shaft. In forms not shown, the mechanism 5 comprises an end stop that limits the rotation angle of the probe 9 in regards of the support 8.

The beam 10 comprises a first proximal end 13 that is connected to the support 8, and a second distal end 14 connected to the probe 9.

The beam 10 is able to buckle to allow shifting from an undeformed position, illustrated in FIG. 5, to a deformed position represented in FIG. 6. The shifting from the undeformed position to the deformed position occurs when the mechanism 5 is subjected to a force exerted by the ossicle 2 Fₒₛₛ, opposite of the force of the sensing tool 1 Fₜₒₒₗ, that is greater than a predetermined threshold force Fora in the beam 10. In the deformed position, the force Fₜₒₒₗ exerted by the sensing tool 1 on the ossicle 2 becomes quasi-constant, irrespective of the deformed position.

The undeformed position is stable. In other words, the beam 10 has the ability to stay in the undeformed position as long as the force exerted by the ossicle 2 Fₒₛₛ is below the predetermined threshold force F_{crit}. Thus, the user can deduce that the measure of the displacement of one ossicle 2 does not have to be yet performed.

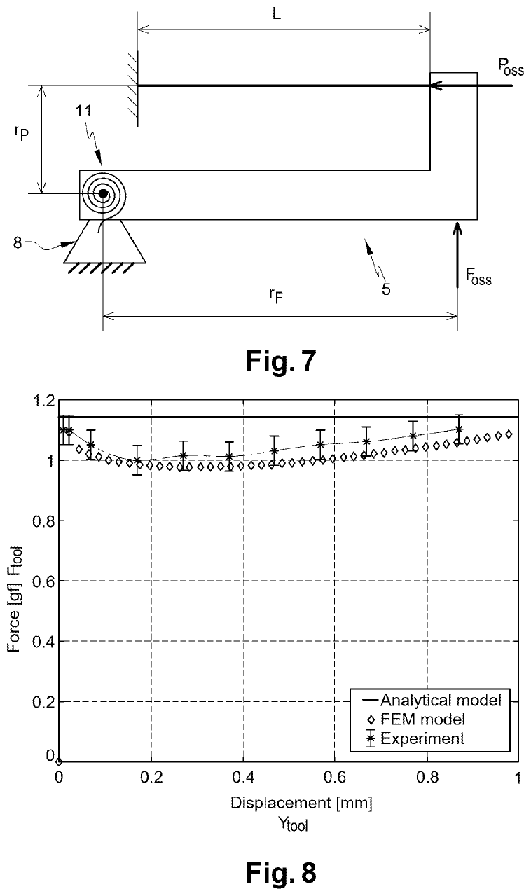

The threshold force F_{crit} can be determined through an analytical model, illustrated in FIG. 7. Such analytical model is a simplification of the mechanism 5 illustrated in FIGS. 2 to 4. When the ossicle 2 applies a force Fₒₛₛ on the probe 9, a compressive load Pₒₛₛ is created within the beam 10. When the compressive force Pₒₛₛ is larger than the Euler's critical load P_{crit}, the beam 10 starts to buckle, and thus the probe 9 rotates. The beam 10 flexural rigidity is EI, E representing the Young's modulus of the material, I representing the quadratic moment of area of the beam 10. The beam 10 in an undeformed position has a length L, the ossicle force Fₒₛₛ on the probe 9 is exerted at a horizontal distance r_{F} of the pivot 11, and the compressive force Fₒₛₛ on the beam 10 is positioned at a vertical distance r_{P} of the flexure pivot 11.

When a force Fₜₒₒₗ is exerted by the sensing tool 1 on the ossicle 2, the ossicles 2 apply a counter-reaction force Fₒₛₛ on the probe 9. When the counter-reaction force Fₒₛₛ is larger than the sensing tool 1 critical load P_{crit}, the beam 10 starts to buckle, and thus the probe 9 rotates.

For the analytical model, the formula of the critical force on the ossicles 2 Fcrit is linked to the Euler's critical load P_{crit} of slender beams with respect to the lever ratio

$$\frac{{r}_{P}}{{r}_{F}} :$$

$${F}_{crit} = {P}_{crit} \frac{{r}_{P}}{{r}_{F}} = \frac{{( 2 π )}^{2} EI}{{L}^{2}} \frac{{r}_{P}}{{r}_{F}}$$

In other word, when force Fₜₒₒₗ exerted by the sensing tool 1 is over the threshold force F_{crit}, the beam 10 buckles, and the sensing tool 1 exerts then a constant load on the ossicle 2 palpated. The mechanism 5 can then be considered as a constant force mechanism or quasi-constant force mechanism.

The beam 10, in the undeformed position is not completely straight, but is very slightly curved (not shown). The curvature of the beam 10 is chosen to control the direction of the buckling of the beam 10. The curvature radius of the beam 10 is the largest value for which a deflection occurs in order the obtain the smallest possible curvature, but sufficient to impact the buckling direction.

The beam 10 has a convex curvature in view of the probe 9. The beam 10 is forced to buckle in the direction of the probe 9. In other word, when the beam 10 gets closer to the probe 9, in one form, the connection part 15 when the beam 10 buckles. A buckling in such a direction enables a force exerted by the sensing tool 1 on the ossicle 2 that is substantially constant.

As shown in FIG. 8, a force-displacement characteristic has been performed to test the mechanism 5. The plot has been obtained by gradually applying a displacement to the probe 9 of the mechanism 5 made of a fused silica while simultaneously measuring the force Fₜₒₒₗ of the end part 17. The force Fₜₒₒₗ remains almost constant, when the beam 10 buckles downward, i.e., towards the probe 9.

In the illustrated forms the probe 9 comprises a connection part 15 arranged between a fixing part 16 and an end part 17.

The fixing part 16 and the connection part 15 extend in a longitudinal direction, and the end part 17 extends in an oblique direction. Such features give a shape of a lever of the probe 9. The probe 9 is able to rotate relative to the support 8 in order to apply an effort to the any of the ossicles 2.

In the illustrated forms, the fixing part 16 comprises a proximal edge 18 that is intended to be in contact with the pivot 11.

In the illustrated forms, the mechanism 5 comprises a clearance 19, located between the connection part 15 that encompasses an upper edge 20 facing the beam 10. The clearance 19 gives space to the beam 10 for buckling, which enables the mechanism 5 to occupy a reduced space when deformed.

The upper edge 20 has a curved shape that has a complementary shape with the beam 10 in a deformed position. The curved shape allows the clearance 19, and by extension the mechanism 5, to have a dimension that is reduced.

In the illustrated forms, the mechanism 5 has a lower edge 21 facing the ossicles 2 when the mechanism 5 is used during the measurement of the stiffness of the ossicles 2.

To ease the contact with the ossicle 2 and ease the palpation, the probe 9 comprises an extreme portion 22, that extends from a distal edge 23 on the prolongation of the end part 17. As such, the force of the tool Fₜₒₒₗ on the ossicle 2 is localized. The precision of palpation is increased and is favorable for the precision of the ossicle 2 mobility measurement. Moreover, the adherence of the mechanism 5 can be assured when the user presses the ossicle 2.

To increase the precision for the application of the force of the tool Fₜₒₒₗ on the ossicle 2, the extreme distal portion 22 defines a tip 24 enabling the probe 9 to adhere to the ossicle 2 that palpation is to be provided.

As an example, the extreme portion 22 has a polygonal shape for example a pyramid shape having a base that corresponds to the distal edge 23, and an apex that protrudes, forming the tip 24.

The mechanism 5 is equipped with a measuring element. As illustrated, the measuring element comprises a plurality of reference points, for example protruding sections 25 protruding from the end part 17 in a transverse direction. Thus, the user can use such reference points as a scale to quantify the displacement performed by the ossicle 2.

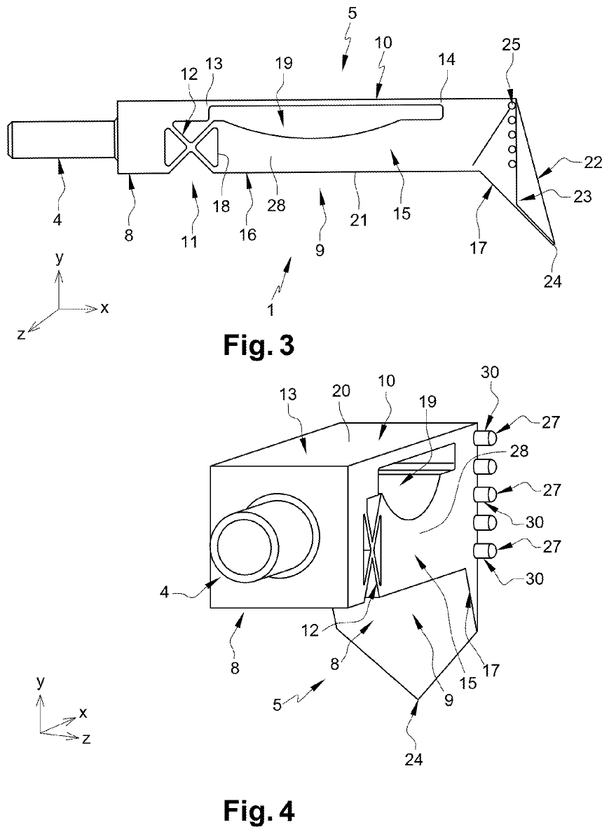

As shown in FIGS. 3 and 4, a protruding section 25 incorporates a cylinder 26 topped by a semi-spherical globe 27. Such protruding section 25 can be monolithic with the mechanism 5 or assembled on the probe 9. In other forms the protruding section 25 integrates a cone.

In the illustrated forms, the probe 9 and the beam 10 define together two lateral surfaces 28, that belong both favorably to the transversal plane XY. The size of the mechanism 5 is reduced. Hence, the palpation does not have a protrusion shape in the transverse direction.

The transverse distance between each lateral surface 28 is approximately 1 mm to limit the size of the mechanism 5. As such, the surgeon is able to insert the sensing tool 1 in the ear canal and obtain a large visual feedback of the middle ear under the operating microscope.

The mechanism 5 is made of a deformable material, that is adapted to the value of the threshold force F_{Crit} desired.

In some forms, the mechanism 5 is made of glass, for example fused silica. Such material is biocompatible and easy to sterilize.

Alternatively, the mechanism 5 can be made of metal, that is stronger than glass and can be easily sterilized.

Alternatively, the mechanism 5 is made of thermoplastic or thermoset plastics.

Alternatively, the mechanism 5 is made of silicon.

It is also possible to use multiple materials and fabrication processes to manufacture the mechanism 5, such as assembly of a metal beam inserted inside a micro-molded plastic mold.

In the following, some manufacturing methods of the mechanism 5 as illustrated are described.

According to a manufacturing method, the mechanism 5 is printed, for example by femto-laser. More precisely, a femto-second laser beam advantageously combined with chemical treatment enables carving in glass a 3D structure, especially fused silica. Femto-laser printing allows the manufacturing of the mechanism 5 with high precision.

According to another manufacturing method, the mechanism 5 is manufactured with a material removal process that can be carried out on material other than glass. Such techniques include electrical discharge machining (EDM).

According to another manufacturing method, the mechanism 5 is made by 3D microprinting or micro-molding, which enables a production in resin for example.

According to another manufacturing method, the mechanism 5 is made by deep reactive ion etching (DRIE), which enables a production in silicon.

In the following, a method of assessment of the mobility of the chain of ossicle via an example of a sensing tool 1 equipped with the above-mentioned mechanism 5 is depicted.

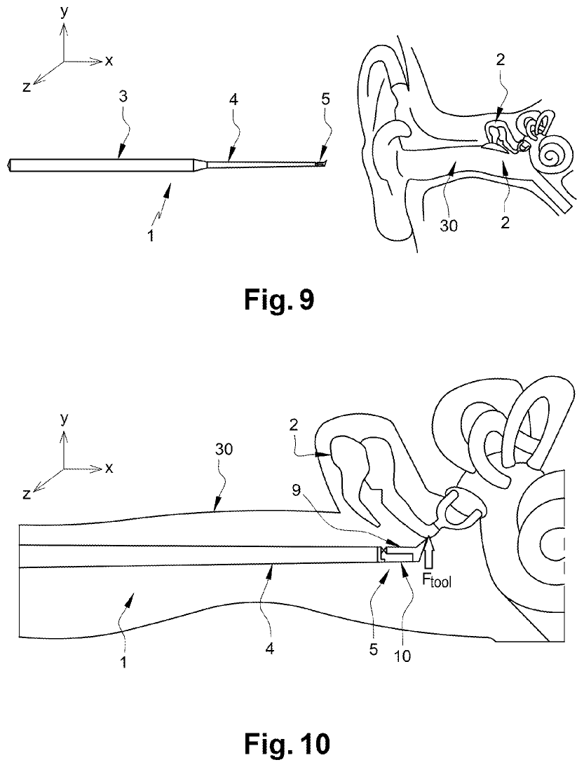

In a step of preparation, illustrated in FIG. 9, the user cuts and folds the eardrum 29 of a patient to leave the ear canal 30 free of obstacles. Such step allows the user to have access to the middle ear.

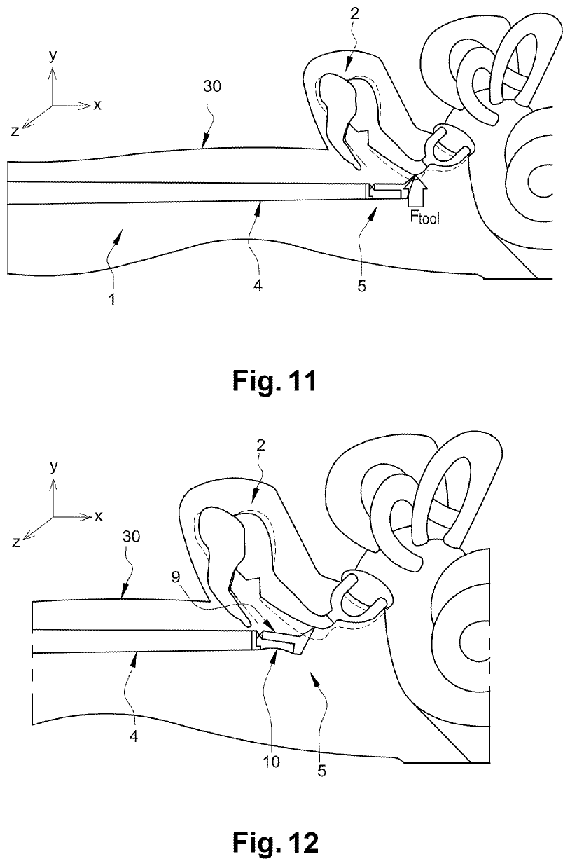

As represented in FIG. 11, the user introduces the sensing tool 1 through the ear canal 30 free of obstacles and positions the mechanism 5 against one of the ossicles 2.

At the time the mechanism 5 is in contact with one of the ossicles 2, the surgeon exerts a pressure load on the handle element 3 of the sensing tool 1 by displacing the sensing tool 1 of an amplitude yₜₒₒₗ, which has the effect of transferring the effort on to the ossicles 2. In such a palpation step, the force exerted by the tool Fₜₒₒₗ can be exerted in various directions. In other forms, the force Fₜₒₒₗ is applied in a vertical direction as illustrated in FIG. 11. Due to the application of the force Fₜₒₒₗ of the sensing tool 1, the palpated ossicle 2 exerts a counterforce Fₒₛₛ on the mechanism 5. When the force Fₜₒₒₗ exerted exceeds the threshold force F_{crit}, then the beam 10 buckles, allowing the mechanism 5 to be in a deformed position, as illustrated in FIG. 12. In such a deformed position, the mechanism 5 exerts a constant pressure on the ossicles 2. As such, the mechanism 5 limits the load applied to the ossicles 2.

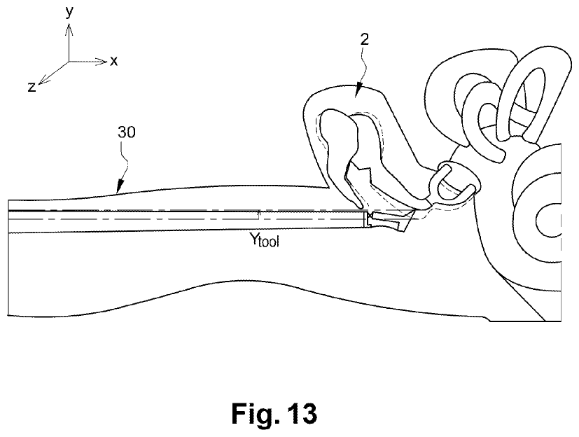

Based on the ossicle stiffness (i.e., reciprocal of the ossicle mobility), the ossicle 2 is displaced by a distance y_{osside} proportionally to the applied load Fₜₒₒₗ. The user measures then the position of the ossicles 2 y_{osside}, for example, with the help of the measuring elements, and the assistance of a microscope (not shown). Such step is represented in FIG. 13.

Knowing the force of application of the mechanism 5 on the ossicle 2, as well as the position of the displaced ossicle 2, the surgeon is able to perform a ratio of these two quantities, and to deduce the mobility of the ossicular chain.

An exemplary method of assessment of the mobility of an ossicle 2 of a person, comprises: a step folding up an eardrum 29, a step of insertion of the sensing tool 1 in the ear canal 30, a step of choice of an ossicle 2 to palpate, the application of an increasing force to the chosen ossicle 2 until the beam 10 reaches the deformed position, and a measurement of the displacement of the ossicle 2.

The assessment of the mobility of the ossicular chain can be repeated with different sensing tools 1 that are constructed in such a way to present a different critical load P_{crit}. As such, the mobility is tested under different values of threshold load F_{crit}, which allows obtaining measurements that are considered reliable.

Alternatively, the measurement is repeated with a single sensing tool 1, for which the critical load P_{crit} is adjusted after each measurement.

The above-depicted method can be applied for a pre-operative assessment, for example to select the appropriate surgery operation.

The method is, for example, pre-operative, enabling to have a confirmation just before making the surgical gestures.

The method can be applied during or for post-operation follow-up, for example to check if the ossicles chain has recovered normal mobility.

In other forms, the mechanism 5, as described above, can be used in other medical applications for which limiting the value of a load on a body is desired.

As an example, the mechanism 5 can be applied for the peeling of the epiretinal membrane of a patient.

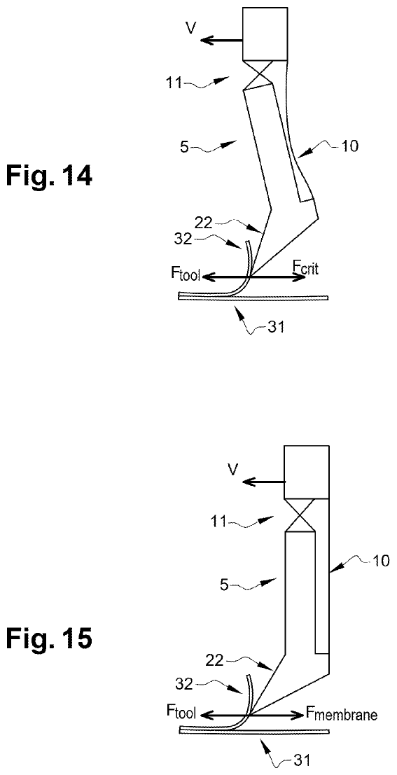

In a second use, illustrated in FIGS. 14 and 15, the mechanism 5 is used in sensing tool 1 intended to remove an ERM. As shown, the eye receiving the intervention is simplified, the ERM covering a retina 31 is represented by a membrane 32.

In a non-illustrated step, after removal of the vitreous gel and substitution with adapted liquid, the mechanism 5 is inserted into the eye.

The end part 17 of the mechanism 5 is positioned against the membrane 32, and as illustrated in FIG. 14, the mechanism 5 is displaced to peel away the membrane 32 from the retina 31. Such displacement is carried out at a speed V in order to apply a sensing force Fₜₒₒₗ against the membrane 32, the membrane 32 applying a reaction force F_{membrane} to the tool 1.

As the mechanism 5 is undeformed, that is illustrated in FIG. 15, the user is informed that the sensing force Fₜₒₒₗ exerted by the sensing tool 1 is below the threshold force F_{crit}, defined to be the maximal harmless force admissible by the retina 31.

When the sensing force Fₜₒₒₗ becomes higher than the threshold force F_{crit}, the beam 10 starts buckling, pivoting the mechanism 5, as shown in FIG. 14.

The surgeon receives the information that the sensing load Fₜₒₒₗ has to be reduced.

Even if the surgeon does not immediately notice that the mechanism 5 is deformed, the sensing force Fₜₒₒₗ is nearly constant and equal to the threshold force F_{crit}. In such a way, the retina 31 of the patient cannot be deformed. In other words, the mechanism 5 has a role of a force limiting mechanism.

An exemplary method of detecting a force during the removal of a membrane 32 from a retina 31 is described. The method comprising a step of positioning the sensing tool 1 against the membrane 32, and the application of an increasing force to peel the membrane 32 until the beam 10 reaches the deformed position to inhibit excessive forces that could harm the patient's retina 31.

The mechanism 5, described above, presents many advantages for example: a limited cost due to the manufacturing process, a compact design, a simple handling, an operation without the use of electric energy, a possibility to get accurate and precise measurements enabling the exploitation of the results by the surgeon, and a possibility of use as constant-force and/or limited force mechanism.

Unless otherwise expressly indicated herein, all numerical values indicating mechanical/thermal properties, compositional percentages, dimensions and/or tolerances, or other characteristics are to be understood as modified by the word “about” or “approximately” in describing the scope of the present disclosure. The modification is desired for various reasons including industrial practice, material, manufacturing, and assembly tolerances, and testing capability.

As used herein, the phrase at least one of A, B, and C should be construed to mean a logical (A OR B OR C), using a non-exclusive logical OR, and should not be construed to mean “at least one of A, at least one of B, and at least one of C.”

The description of the disclosure is merely exemplary in nature and, thus, variations that do not depart from the substance of the disclosure are intended to be within the scope of the disclosure. Such variations are not to be regarded as a departure from the spirit and scope of the disclosure.

What is claimed is:

1. A micromachined mechanism for a sensing tool for a medical or surgical use, the micromachined mechanism comprising:
a support;
a probe pivotally mounted with respect to the support; and
a beam connected to the support and the probe, wherein the probe is configured to apply a load on a body and to be subjected to a counter-reaction contact force exerted by the body in reaction to the load, and
wherein the beam is configured to shift from an undeformed position to a deformed position when the contact force of the body is greater than a predetermined threshold force for which the beam buckles.

2. The mechanism according to claim 1, wherein the probe is pivotally mounted with respect to the support via a flexure pivot.

3. The mechanism according to claim 1, wherein the probe is pivotally mounted with respect to the support via a pivot connection comprising a pin, and a torsion spring.

4. The mechanism according to claim 1, wherein the mechanism is monolithic.

5. The mechanism according to claim 1, wherein the mechanism is made of metal.

6. The mechanism according to claim 1, wherein the mechanism is made of silicon.

7. The mechanism according to claim 1, wherein the mechanism is made of glass.

8. The mechanism according to claim 1, wherein the mechanism is made of plastic.

9. The mechanism according to claim 1, wherein in the undeformed position, the beam extends along a slightly curved trajectory, the curved trajectory being convex relative to the probe.

10. The mechanism according to claim 1, further comprising an extreme portion comprising a tip configured to be in contact with the body.

11. The mechanism according to claim 1, wherein the probe comprises a connection part and an extreme portion, the extreme portion protruding from the connection part.

12. The mechanism according to claim 1, further comprising an end part comprising a graduation element, the graduation element protruding from the end part.

13. The mechanism according to claim 12, wherein the graduation element comprises a three-dimensional solid arranged along a surface of the end part.

14. The mechanism according to claim 13, wherein the three-dimensional solid is a cylinder.

15. The mechanism according to claim 1, wherein the mechanism is adapted for a sensing tool configured to palpate an ossicle, and wherein the body is the ossicle.

16. The mechanism according to claim 1, wherein the mechanism is adapted for a sensing tool configured to remove a membrane from a retina, the body being the membrane.

17. A sensing tool comprising:
a handle element;
a holding element, placed at one end of the handle element; and
a mechanism according to claim 1, the mechanism being rigidly connected with the holding element.

18. A method of manufacturing the mechanism of claim 1, the method comprising a step of formation, the step of formation being carried out by femto-laser printing, three dimensional printing, molding, electrical discharge machining, deep reactive ion etching, or a combination thereof.
